(19) United States
(12) Patent Application Publication
(10) Pub. No.: US 2012/0143022 A1
(43) Pub. Date: Jun. 7, 2012

(54) PHYSIOLOGICAL SIGNAL DETECTION SYSTEM CAPABLE OF SHOWING EMOTIONS, DEVICE AND EMOTIONAL DISPLAY METHOD

(76) Inventors: Kuan-Rong LEE, Chiayi City (TW); Yau-Hwang Kuo, Tainan City (TW); Ying-Hsu Chen, Kaohsiung City (TW)

(21) Appl. No.: 12/976,620

(22) Filed: Dec. 22, 2010

(30) Foreign Application Priority Data
Dec. 2, 2010 (CN) 201010579947.4

(51) Int. Cl. A61B 5/00; A61B 5/021; A61B 5/01; A61B 5/0295; A61B 5/026; A61B 5/024

(52) U.S. Cl. 600/307; 600/504; 600/508; 600/549; 600/322; 600/500

(57) ABSTRACT

A physiological signal detection system and device capable of showing emotions and an emotional display method are provided. The system includes: a display unit; a storage unit, for storing a plurality of conditions of emotional index; a physiological signal detection unit, for acquiring at least one physiological signal; a physiological signal recognition unit, for recognizing and filtering the at least one physiological signal; an emotional index operation unit, for generating a value of emotional index according to the at least one physiological signal; and a processing unit, for comparing the conditions of emotional index according to the value of emotional index to generate a comparison result and displaying a display signal corresponding to the value of emotional index on the display unit according to the comparison result.

### CROSS-REFERENCE TO RELATED APPLICATION

This application claims the benefit of China Patent Application No. 201010579947.4, filed on Dec. 2, 2010, which is hereby incorporated by reference for all purposes as if fully set forth herein.

### BACKGROUND OF THE INVENTION

1. Field of Invention

The present invention relates to a physiological signal detection system and device capable of showing emotions and an emotional display method, and more particularly relates to a physiological signal detection system and device capable of analyzing emotions according to a physiological signal and showing the emotions and a display method thereof.

2. Related Art

In regard with the fact that modern people pays more attention to their physical status and as consequently demands for the physical monitoring devices, especially in the quick pace of modem society, it becomes more and more difficult to ask for a leave to go to hospital. In addition, the phenomenon of aging society in current years, the demand for home health care grows quickly, and thus the physiological signal monitoring device is put forward for satisfying the needs for monitoring physical conditions anytime.

However, even if a variety of wireless, portable, handheld physiological signal monitoring devices have been developed after the continuous improvement and vigorous development, which indeed overcomes some defects of the large-sized monitoring devices placed beside the patient bed and is convenient to take and use, the general physiological signal detection system or device mainly detects the physiological signals of the user and does not provide the emotion recognition and display functions, so the psychological status cannot be taken care. In the current medical system, the psychological health and consultant service are greatly valued. If the emotion detection and display functions may be added into the physiological signal detection equipment/device, it helps the user to know the emotional status, and further, the emotional status of the user may be sent to the medical staffs to keep a record, so that the medical practitioners may observe the emotion of the user to take care or conduct physiological counseling at a proper time. Hence, there is a need for a monitoring device and system which is chic, easy to move and meanwhile capable of recording a complete physiological signal detection of a user in a measurement period and also meets the requirements for detection/display of the emotional status.

Furthermore, the current hospital equipment or many psychological monitoring devices (e.g., a healthcare homebox) generally used at home of the user only have the physiological signal detection, read or inspection functions. Therefore, it is rather important to integrate the existing physiological signal detection equipments to provide further emotion recognition and display functions.

### SUMMARY OF THE INVENTION

A physiological signal detection system and device capable of showing emotions and a detection display method are disclosed in the present invention, which combines a variety of physical sensing device techniques and emotion deduction techniques to enable a general physiological signal detection device to detect and recognize an emotional status of a user.

A physiological signal detection system capable of showing emotions is disclosed in the present invention, which includes: a display unit; a storage unit, for storing a plurality of conditions of emotional index; a physiological signal detection unit, for acquiring at least one physiological signal; a physiological signal recognition unit, for recognizing and filtering the at least one physiological signal; an emotional index operation unit, for generating a value of emotional index according to the at least one physiological signal; and a processing unit, for comparing the conditions of emotional index according to the value of emotional index to generate a comparison result, and displaying a display signal corresponding to the value of emotional index on the display unit according to the comparison result.

A physiological signal detection device capable of showing emotions is disclosed in the present invention, which includes: a casing; a display unit, disposed outside the casing; a physiological signal detection unit, disposed outside the casing, for acquiring at least one physiological signal; a storage unit, disposed inside the casing, for storing a plurality of conditions of emotional index; and a processor, disposed inside the casing and coupled to the storage unit, a physiological signal detector and the display unit, for recognizing and filtering the at least one physiological signal, generating a value of emotional index according to the at least one physiological signal, comparing the conditions of emotional index according to the value of emotional index to generate a comparison result and displaying a display signal corresponding to the value of emotional index on the display unit according to the comparison result.

An emotional display method using physiological signal detection is disclosed, which is adapted for an electronic equipment which at least includes a storage module for storing a plurality of conditions of emotional index and a display unit. The method includes:

acquiring at least one physiological signal by a physiological signal detection unit; recognizing and filtering the at least one physiological signal by a physiological signal recognition unit; generating a value of emotional index by a processing unit according to the at least one physiological signal, comparing the conditions of emotional index according to the value of emotional index to generate a comparison result; and displaying a display signal corresponding to the value of emotional index on the display unit according to the comparison result.

A computer program product is further disclosed in the present invention, which is provided for an electronic equipment to read and execute the above emotional display method using physiological signal detection, and the processing flows may refer to the above description and the details will not be repeated herein again.

In an embodiment of the present invention, the above physiological signal includes at least one selected from a signal of body temperature, a signal of sweat gland secretion, a signal of blood flow rate and a signal of heat rate.

In an embodiment of the present invention, the above signal of body temperature uses a thermometer to measure a body temperature.

In an embodiment of the present invention, the above signal of sweat gland secretion is collected by a skin-conductance-response (SCR) machine.

In an embodiment of the present invention, the above signal of blood flow rate uses a photoplethysmography scanner to collect a blood flow rate.

In an embodiment of the present invention, the above signal of heat rate uses an electrocardiographic patch to collect a variation ratio of wrist pulse.

According to the present invention, sensing devices of a body temperature, pulse heart rate, degree of sweat gland secretion and blood flow rate etc. (physiological signal detection) may be used to deduce a current emotional index of the user and express the emotional index into the measured emotional status instantly with a graph, light or sound, thereby enabling the user or health care to know the current emotion of the user.

### BRIEF DESCRIPTION OF THE DRAWINGS

The present invention will become more fully understood from the detailed description given herein below for illustration only, and thus are not limitative of the present invention, and wherein:

FIG. 1 is a schematic block view of elements of a health care detection display system of the present invention;

FIG. 2 is a flow chart of a health care detection display method of the present invention;

FIG. 3 is a physiological signal detection device capable of showing emotions of the present invention;

FIG. 4A is a schematic view of a health care detection device according to an embodiment of the present invention;

FIG. 4B is a schematic view of a pleasure emotion of the person under detection displayed by a health care detection device according to an embodiment of the present invention; and

FIG. 4C is a schematic view of a pain emotion of the person under detection displayed by a health care detection device according to an embodiment of the present invention.

### DETAILED DESCRIPTION OF THE INVENTION

Hereinafter, the details of the embodiments of the present invention will be illustrated with reference to the drawings.

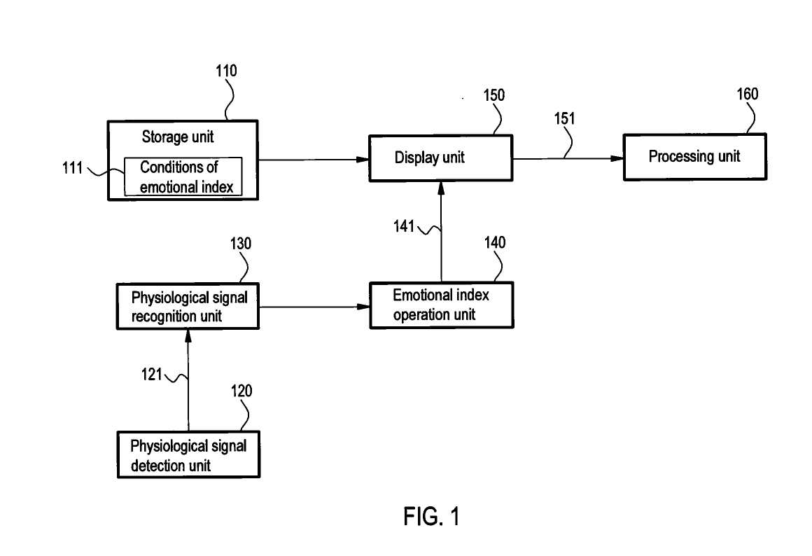

FIG. 1 is a schematic block view of system elements of the present invention. Referring to FIG. 1, the system includes a storage unit 110, a physiological signal detection unit 120, a physiological signal recognition unit 130, an emotional index operation unit 140, a processing unit 150 and a display unit 160. The storage unit 110 stores a plurality of conditions of emotional index 111. The physiological signal detection unit 120 acquires at least one physiological signal 121. The physiological signal recognition unit 130 recognizes a physiological signal 121 and filters an interference signal in the physiological signal 121. The emotional index operation unit 140 generates a value of emotional index 141 according to the physiological signal 121. The processing unit 150 compares the conditions of emotional index 111 according to the value of emotional index 141 to generate a comparison result 151, and displays a display signal corresponding to the value of emotional index 141 on a display unit 160 according to the comparison result 151.

The system of the present invention may be built on a computer or a portable device (for example, a personal computer, a laptop computer, a notebook computer, PDA, etc.), and is implemented to be linked to the physiological signal detection unit, may also be implemented to be combined with a physiological signal detection system/device (implemented to be, a health care kit, a physiological signal recorder, remote home physiological signal monitoring system, etc), and may also be implemented to be a dedicated system.

In the embodiment, the physiological signal may be a signal of body temperature, a signal of sweat gland secretion, a signal of blood flow rate and a signal of heat rate. Correspondingly, the physiological signal recognition unit 130 may be any device or detector for measuring the above physiological signals, and transmits the physiological signal in a wired communication, a wireless communication or RS232 connection manner to the emotional index operation unit 140, for example, a thermometer, a heart rate sensor, a SCR machine or a photoplethysmography scanner.

In the embodiment, the signal of body temperature uses a thermometer to measure a body temperature of a human body, for example, uses a finger thermometer to measure a temperature of fingers, an ear measures thermometer to measure a temperature of ears, a forehead thermometer to measure a temperature of the forehead and the like.

In the embodiment, the signal of heat rate may be measured by a heart rate sensor, for example, a heart rate detector and an electrocardiographic patch for collecting a variation ratio of wrist pulse, a heat rate monitor which uses a finger sensor to measure the heart rate or the heart rate monitor instrument. The signal of heat rate captured techniques includes time domain analysis and frequency domain analysis.

1. Characteristic values captured in the time domain analysis include:
(1) SDNN: a standard difference of all normal-to-normal RR intervals (NN): in a unit of ms.
(2) RMSSD: a root mean square of successive differences between adjacent normal NN intervals: a unit of ms.
(3) NN50: the number of pairs of successive NNs that differ by more than 50 ms.
(4) PNN50: the proportion of NN50 divided by total number of NNs.

2. Characteristic values captured in the frequency domain analysis include:
(1) High frequency (HF, 0.15 to 0.4 Hz): related to respiration, the main peak frequency is respiratory frequency.
(2) Low frequency (LF, 0.04 to 0.15 Hz): reflective control from the pressure sensor, related to homeostasis.
(3) Very low frequency (VLF, 0 to 0.04 Hz): 0 to 0.04 Hz, rhythm of diastolic and systolic tension of the peripheral vessels, related to heat regulation.

In the embodiment, the signal of sweat gland secretion uses a SCR machine to the degree of sweat gland secretion of a middle finger and a ring finger. The SCR machine is for example, a physical feedback skin conductance monitor for collecting SCR (Skin Conductance Responce) to the external or internal stimulation.

The characteristic values of the degree of sweat gland secretion are captured according to time-domain analysis and the characteristic values are calculated by, for example:

w1*SDNN+w2*RMSSD+w3*NN50+w4*PNN50

where w1, w2, w3, w4 are weighted parameters set depending on the personal physical characteristic of the user or determined by an experience value/suggested value from the medical practitioner.

In the embodiment, the signal of blood flow rate uses a photoplethysmography scanner (e.g., a photoplethysmograph) to collect a blood flow rate of a pointer finger. The blood flow rate is captured according to time-domain analysis, a variation ratio is calculated every other 1 ms, and when the variation ratio exceeds a personal reference value v1, the variation ratio is set to 1, or otherwise is set to 0. The times when the variation ratio is 1 is converted to a frequency value. If the frequency value exceeds v2 (that is, the high frequency), the characteristic value is set to 10. If the frequency value exceeds v3 and is lower than v2 (that is, the low frequency), the characteristic value is set to 5. If the frequency value is lower than v3 (that is, the very low frequency), the characteristic value is set to 0. The v1,v2,v3 are reference vale parameters set depending on personal characteristic of the user or determined by an experience value/suggested value from the medical practitioner.

The storage unit 110 stores a plurality of conditions of emotional index 111, which may be a device or element generally used for storing data, e.g., a hard drive, a solid state disk, a flash memory or a portable disk etc., and the type of the storage unit 110 is not limited herein. The conditions of emotional index 111 may be designed according to the practical requirement or determined according to a general experience. For example, the emotion is divided into four index conditions, namely joy, anger, sorrow and happiness, and each condition of emotional index defines a data range of at least one physiological signal, e.g., regarding the condition of emotional index of “anger”, the heart rate may quickly increase when above a certain value and the characteristic value of the degree of sweat gland secretion is in a certain range. The conditions of emotional index 111 may also be a value of emotional index and a relevant data corresponding to the emotion, and different score ranges are set and respectively correspond to different emotional status, e.g., a score below 8 indicates the emotion is stable, a score between 9 to 14 indicates the emotion is unstable, and a score between 15 to 18 indicates the pressure and loads have reached the critical point etc.

The emotional index operation unit 140 generates a value of emotional index 141 according to the physiological signal 121. A variety of emotional index deduction mechanisms, for example, a Support Vector Machines (SVM) is used to collect a plurality of sample physiological signal and the corresponding values of emotional index in advance and after comparing and classifying, establish an emotion database or a classification model. After that, when input or measure a physiological signal of the user, the mechanism generates a corresponding value of emotional index according to the emotion database or the classification model. Or, an emotion analysis calculation formula is provided, and the detected physiological signal is input for calculation, thereby obtaining the corresponding value of emotional index.

The processing unit 150 compares the conditions of emotional index 111 according to the value of emotional index 141 to generate a comparison result 151. As described above, different score ranges are set for the conditions of emotional index and respectively correspond to different emotional status. When the corresponding value of emotional index generated is 6, the comparison result is lower than 8, the emotion is determined to be stable according to the conditions of emotional index 111.

The emotional index operation unit 140 and the processing unit 150 may also be integrated in the same unit, which may be realized by using a processor/operator to load a relevant program code, or the relevant program code is burned into a specific chip or processor to make the chip or processor become the emotional index operation unit 140 and the processing unit 150.

Accordingly, the user may put palm on the physiological signal recognition unit 130 of the present invention system to sense the physiological signal 121. For example, the user put wrist on a wrist support seat of the health care kit built with the system of the present invention, and the physiological signal recognition unit 130 may sense the pulse and heart rate information. The heart rate detector uses the electrocardiographic patch to collect the variation ratio of wrist pulse serving as the reference for measuring the heart rate (measuring time takes one minute as the sampling time). Other interference signals may be obtained when the physiological signal 121 is acquired, so the physiological signal recognition unit 130 may further filter the abnormal physiological signal 121 so as to acquire the effective physiological signal 121 to improve the accuracy of the health care detection display system in analyzing the physiological signal 121.

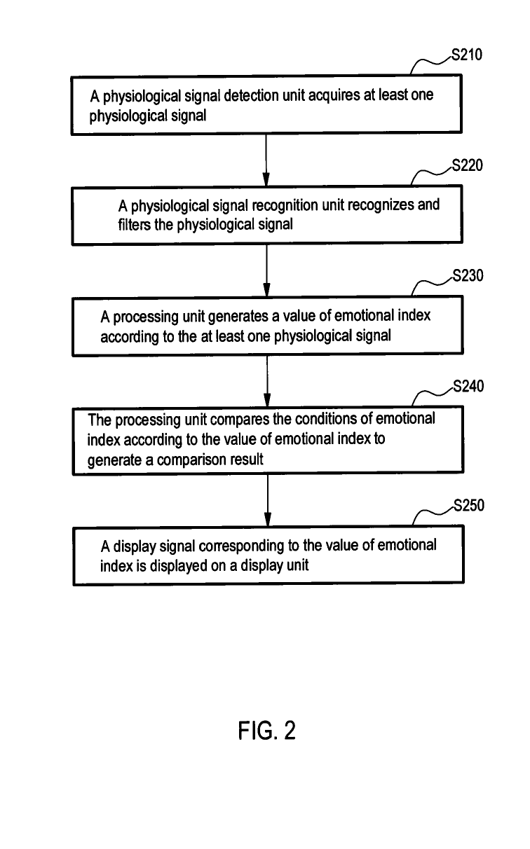

FIG. 2 is a flow chart of an emotional display method using physiological signal detection according to the present invention. The method is adapted for an electronic equipment and the electronic equipment at least includes a storage module for storing a plurality of conditions of emotional index and a display unit. The method includes the following steps.

Step S210: Firstly, a physiological signal detection unit acquires at least one physiological signal. In the embodiment, the physiological signal includes at least one signal of body temperature, a signal of sweat gland secretion, a signal of blood flow rate and a signal of heat rate.

Step S220: A physiological signal recognition unit recognizes and filters the physiological signal. In the embodiment, other interference signals may be obtained when acquiring the physiological signal, so the physiological signal recognition unit may filter the abnormal physiological signal to acquire the effective physiological signal.

Step S230: Then, a processing unit generates a value of emotional index according to the at least one physiological signal.

Step S240: The processing unit compares the conditions of emotional index according to the value of emotional index to generate a comparison result.

Step S250: Finally, a display signal corresponding to the value of emotional index is displayed on a display unit.

The detailed techniques of the emotional display method using physiological signal detection of the present invention are described as above and will not be repeated herein again. The emotional display method using physiological signal detection may also b realized by a computer program product, and the computer program product is provided for an electronic equipment (for example, a computer, PDA, a health care kit etc.) to read and execute. The electronic equipment at least includes a storage module for storing a plurality of conditions of emotional index and a display unit. The method includes the steps as described above: acquiring at least one physiological signal by a physiological signal detection unit; recognizing and filtering the at least one physiological signal by a physiological signal recognition unit; generating a value of emotional index by a processing unit according to the at least one physiological signal, comparing the conditions of emotional index according to the value of emotional index to generate a comparison result; and displaying a display signal corresponding to the value of emotional index on the display unit according to the comparison result.

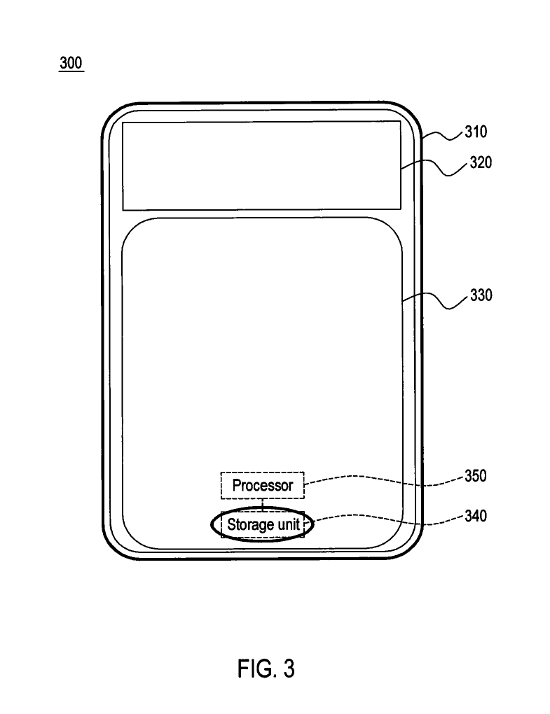

FIG. 3 illustrates a physiological signal detection device capable of showing emotions of the present invention. Referring to FIG. 3, the physiological signal detection device capable of showing emotions 300 mainly includes a casing 310, a display unit 320, a physiological signal detection unit 330, a storage unit 340 and a processor 350. The display unit 320 is disposed outside the casing 310, for example, a LCD screen, a set of display lamps or a sound player, for displaying/playing the relevant information. The physiological signal detection unit 330 is disposed outside the casing 310, for acquiring at least one physiological signal, for example, the thermometer, the heart rate sensor, the SCR machine, or the photoplethysmography scanner. The physiological signal detection unit 330 may also be designed to be an independent unit, or is connected to the device 300 via a wired or wireless communication network. The storage unit 340 is disposed inside the casing 310, for storing a plurality of conditions of emotional index. The processor 350 is disposed inside the casing 310 and coupled to the storage unit 340, the physiological signal detector 330 and the display unit 320. The processor 350 mainly functions to generate a value of emotional index according to the physiological signal, compare the conditions of emotional index according to the value of emotional index to generate a comparison result, and thereafter display a corresponding display signal on the display unit 320 according to the comparison result. The detailed techniques of the physiological signal detection device capable of showing emotions are described above and will not be repeated herein again.

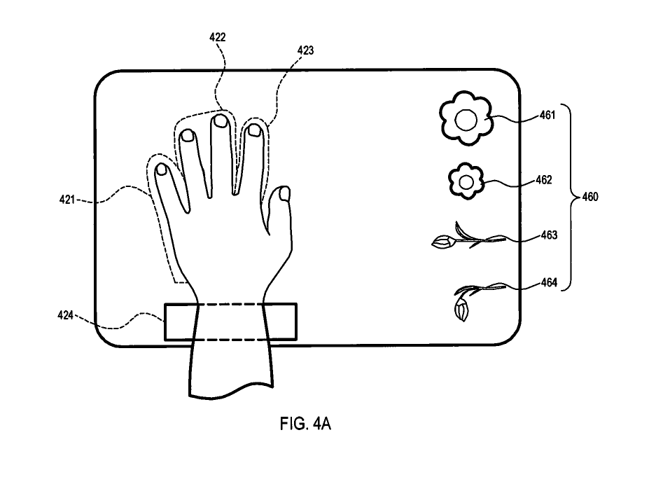

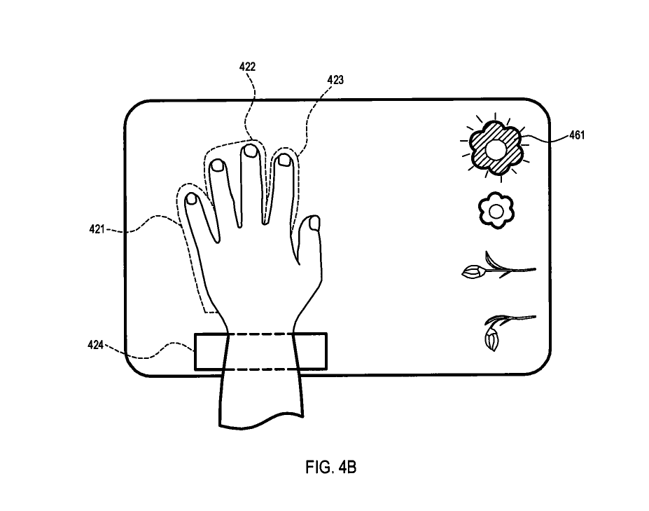

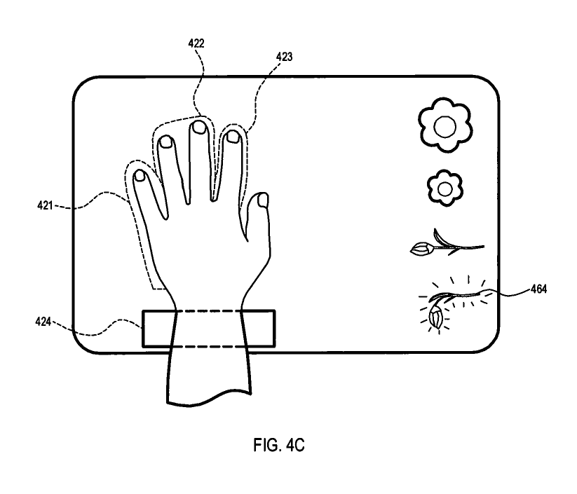

Referring to FIG. 4A to FIG. 4C, FIG. 4A is a schematic view of a physiological signal detection device capable of showing emotions according to an embodiment of the present invention. FIG. 4B is a schematic view of a pleasure emotion of the user displayed by a physiological signal detection device capable of showing emotions according to another embodiment of the present invention. FIG. 4C is a schematic view of a pain emotion of the user displayed by a physiological signal detection device capable of showing emotions according to yet another embodiment of the present invention. In FIG. 4A, the physiological signal detection unit is disposed on the plane of the casing to allow the user to put palm thereon, and corresponding to different finger positions, respectively a signal of body temperature detection unit 421, a signal of sweat gland secretion detection unit 422, a signal of blood flow rate detection unit 423 and a signal of heat rate detection unit 424 are arranged. In this embodiment, the display unit is a plurality of display graphs located on sides of the casing, and according to the comparison result, the lamp for one display graph is lit on. The graphic display of different flower forms in the emotion display unit 460 respectively represents the emotion analysis results of joy, anger, sorrow, happiness of the one under observation. For example, a big flower 461 represents the user is joyful (happiness), a small flower 462 represents the user is happy (joy), a flower bud 463 represents the user is not in mood (anger), and a wilted flower 464 represents the user is depressed (sorrow). Whereby, when the emotion of the user can be measured and displayed by the device, so that the user or other people may know the emotional status through different flowers.

Furthermore, in addition to the display graph of flowers for showing the emotion of the one under observation, a display screen may be used to display letters/graphs, different light effects may be used or different tones and music may be played to express the detected result.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A physiological signal detection system capable of showing emotions, comprising:
a display unit;
a storage unit, for storing a plurality of conditions of emotional index;
a physiological signal detection unit, for acquiring at least one physiological signal;
a physiological signal recognition unit, for recognizing and filtering the at least one physiological signal;
an emotional index operation unit, for generating a value of emotional index according to the at least one physiological signal; and
a processing unit, for comparing the conditions of emotional index according to the value of emotional index to generate a comparison result and displaying a display signal corresponding to the value of emotional index on the display unit according to the comparison result.

2. The physiological signal detection system capable of showing emotions according to claim 1, wherein the physiological signal is one selected from a group consisting of a signal of body temperature, a signal of sweat gland secretion, a signal of blood flow rate and a signal of heart rate.

3. The physiological signal detection system capable of showing emotions according to claim 2, wherein the signal of body temperature uses a thermometer to measure a body temperature.

4. The physiological signal detection system capable of showing emotions according to claim 2, wherein the signal of sweat gland secretion is collected by a skin-conductance-response (SCR) machine.

5. The physiological signal detection system capable of showing emotions according to claim 2, wherein the signal of blood flow rate uses a photoplethysmography scanner to collect a blood flow rate.

6. The physiological signal detection system capable of showing emotions according to claim 5, wherein the signal of blood flow rate is captured according to time-domain analysis, a variation ratio is calculated every other 1 ms, and when the variation ratio exceeds a personal reference value v1, the variation ratio is set to 1, or otherwise is set to 0.

7. The physiological signal detection system capable of showing emotions according to claim 2, wherein the signal of heart rate uses an electrocardiographic patch to collect a variation ratio of wrist pulse.

8. A physiological signal detection device capable of showing emotions, comprising:
a casing;
a display unit, disposed outside the casing;
a physiological signal detection unit, disposed outside the casing, for acquiring at least one physiological signal;
a storage unit, disposed inside the casing, for storing a plurality of conditions of emotional index; and
a processor, disposed inside the casing and coupled to the storage unit, a physiological signal detector and the display unit, for recognizing and filtering the at least one physiological signal, generating a value of emotional index according to the at least one physiological signal, comparing the conditions of emotional index according to the value of emotional index to generate a comparison result and displaying a display signal corresponding to the value of emotional index on the display unit according to the comparison result.

9. An emotional display method using physiological signal detection, adapted for an electronic equipment, wherein the electronic equipment at least comprises a storage module for storing a plurality of conditions of emotional index and a display unit, the method comprising:
acquiring at least one physiological signal by a physiological signal detection unit;
recognizing and filtering the at least one physiological signal by a physiological signal recognition unit;
generating a value of emotional index by a processing unit according to the at least one physiological signal, and comparing the conditions of emotional index according to the value of emotional index to generate a comparison result; and
displaying a display signal corresponding to the value of emotional index on the display unit according to the comparison result.

10. The emotional display method using physiological signal detection according to claim 9, wherein the physiological signal is one selected from a group consisting of a signal of body temperature, a signal of sweat gland secretion, a signal of blood flow rate and a signal of heat rate.

11. The emotional display method using physiological signal detection according to claim 10, wherein the signal of body temperature uses a thermometer to measure a body temperature.

12. The emotional display method using physiological signal detection according to claim 10, wherein the signal of sweat gland secretion is collected by a skin-conductance-response (SCR) machine.

13. The emotional display method using physiological signal detection according to claim 10, wherein the signal of blood flow rate uses a photoplethysmography scanner to collect a blood flow rate.

14. The emotional display method using physiological signal detection according to claim 13, wherein the signal of blood flow rate is captured according to time-domain analysis, a variation ratio is calculated every other 1 ms, and when the variation ratio exceeds a personal reference value v1, the variation ratio is set to 1, or otherwise is set to 0.

15. The emotional display method using physiological signal detection according to claim 10, wherein the signal of heat rate uses an electrocardiographic patch to collect a variation ratio of wrist pulse.

16. A computer program product, for an electronic equipment to read and execute the emotional display method using physiological signal detection, wherein the electronic equipment at least comprises a storage module for storing a plurality of conditions of emotional index and a display unit, the method comprising:
acquiring at least one physiological signal by a physiological signal detection unit;
recognizing and filtering the at least one physiological signal by a physiological signal recognition unit;
generating a value of emotional index by a processing unit according to the at least one physiological signal, and comparing the conditions of emotional index according to the value of emotional index to generate a comparison result; and
displaying a display signal corresponding to the value of emotional index on the display unit according to the comparison result.
